(19) United States
(12) Patent Application Publication
(10) Pub. No.: US 2025/0099529 A1
(43) Pub. Date: Mar. 27, 2025

(54) COMPOSITION FOR IMPROVING IMMUNITY OF COMPANION ANIMALS

(71) Applicant: Tae Yoon KIM, Yangju-si (KR)

(72) Inventor: Tae Yoon KIM, Yangju-si (KR)

(21) Appl. No.: 18/784,102

(22) Filed: Jul. 25, 2024

(30) Foreign Application Priority Data
Sep. 25, 2023 (KR) 10-2023-0128413

(51) Int. Cl. A61K 36/8998; A61K 31/122; A61K 31/198; A61K 31/202; A61K 31/7028; A61K 31/717; A61K 33/06; A61K 33/30; A61K 35/618; A61K 35/644; A61K 36/05; A61K 36/062; A61K 36/07; A61K 36/68; A61K 36/899; A61K 38/06

(52) U.S. Cl. CPC A61K 36/8998; A61K 31/122; A61K 31/198; A61K 31/202; A61K 31/7028; A61K 31/717; A61K 33/06; A61K 33/30; A61K 35/618; A61K 35/644; A61K 36/05; A61K 36/062; A61K 36/07; A61K 36/68; A61K 36/899; A61K 38/063; A61K 2236/15; A61K 2236/17; A61K 2236/19

(57) ABSTRACT

A composition for improving immunity of companion animals, includes barley sprout extract powder, broccoli sprout extract powder, propolis, omega-3 fatty acid, zeolite powder, arginine powder, glutathione powder, chlorella powder, green leaf mussel extract powder, cranberry powder, shiitake powder, Sparassis crispa powder, Rich-Soybean paste power, brown rice (unpolished rice) enzyme powder, Kamut enzyme powder, plantago seed (psyllium) husk powder, Aspergillus oryzae powder, Monascus pilosus powder, Coenzyme Q-10 powder, zinc powder, N-acetylglucosamine, disodium EDTA and croscarmellose sodium as active ingredients, and enhances proliferation ability of peripheral blood mononuclear cells (PBMCs) through edible feeding to increase immunity, thereby contributing to health improvement.

### CROSS REFERENCE TO RELATED APPLICATION

This application claims priority to Korean Patent Application No. 10-2023-128413 (filed on Sep. 25, 2023), which is hereby incorporated by reference in its entirety.

### BACKGROUND

The present invention relates to a composition for improving immunity of companion animals, and more particularly, to a composition for improving immunity of companion animals, which includes barley sprout extract powder, broccoli sprout extract powder, propolis, omega-3 fatty acid, zeolite powder, arginine powder, glutathione powder, chlorella powder, green leaf mussel extract powder, cranberry powder, shiitake powder, Sparassis crispa powder, Rich-Soybean paste power, brown rice (unpolished rice) enzyme powder, Kamut enzyme powder, plantago seed (psyllium) husk powder, Aspergillus oryzae powder, Monascus pilosus powder, Coenzyme Q-10 powder, zinc powder, N-acetylglucosamine, disodium EDTA and croscarmellose sodium as active ingredients, and enhances proliferation ability of peripheral blood mononuclear cells (PBMCs) through edible feeding to increase immunity, thereby contributing to health improvement.

Pets refer to animals that people like, keep close to and protect, and include dogs, cats, birds, goldfish and the like.

Among them, some pets, such as dogs and cats, are expanding their roles as companion animals that live with their owners and share emotional sympathy in a personalized modern society. In recent years, types of the companion animals are diversifying, for example, include parrots, hedgehogs, rabbits and hamsters as well as the dogs and cats, and related industries are also developing rapidly.

For example, food or snacks for companion animals are not simply a means of supplying nutrients, but contain various ingredients to improve the health of the companion animals, as well as products with improved texture or flavor according to preference.

Meanwhile, similar to the human body, it is also known in the companion animals that occurrence of different diseases is closely related to activity of intestinal beneficial bacteria.

For example, the intestinal bacteria are microorganisms distributed on the intestinal epithelium to live in symbiosis, contribute to metabolic functions, and perform interaction with an immune system, protective function against pathogen, etc.

Such intestinal bacteria may be divided into beneficial bacteria and harmful bacteria, wherein the harmful bacteria generate many toxins and bodily wastes harmful to the intestine to collapse intestinal environment, which in turn may cause different diseases.

On the other hand, the intestinal beneficial bacteria facilitate active bowel movement, suppress harmful bacteria, and are helpful for reinforcing immunity.

Among such intestinal beneficial bacteria, representative ones may include, for example, lactobacillus, bifidobacterium, enterococcus facecium, lactococcus or the like.

Therefore, it tends to constantly increase interest for development of technologies relevant to activation of intestinal beneficial bacteria directly relating to health improvement of the companion animals.

In particular, according to researches, it has been reported that the activity of such intestinal beneficial bacteria have influence on increase in peripheral blood mononuclear cell (PBMC) lymphoids, thereby contributing to immune enhancement.

In this regard, Korean Patent Registration No. 10-2463908 (Nov. 1, 2022) discloses snacks for companion animals for improving intestinal health and a manufacturing method thereof, with a technique in which any one meat selected from the group consisting of duck meat, chicken meat, whole quail meat, tendon, and ox leg, and any one or two or more selected from oatmeal, sweet potato, carrot, paprika, broccoli, king oyster mushroom and parsley are mixed and pulverized; after mixing the pulverized product, olive oil is added thereto; sweet pumpkin, blueberry or milk is added to the mixture, followed by adding aged liquid and introducing fermentable microorganisms including lactic acid bacteria (‘lactobacillus’) or others to increase beneficial bacteria.

Further, Korean Patent Laid-Open Publication No. 10-2023-0083178 (Jun. 9, 2023) discloses a composition for improving palatability and intestinal health of companion animals including sunflower lecithin, with a technique in which beet powder obtained after drying the roots of beets with hot air and carrot powder obtained after drying the roots of carrots with hot air are further included to promote the growth of intestinal beneficial bacteria; a technique in which kelp powder obtained after drying kelp with hot air is further included to use an alginic acid ingredient of the kelp to adsorb and discharge wastes in the intestine; a technique in which hydrolyzed yeast extract powder is further included to promote digestion in the intestine; and a technique in which thyme extract powder obtained by extracting thyme with ethanol is further included to adjust a moisture content of feces in the intestine, thereby maintaining the hardness and viscosity of the feces.

### PRIOR ART DOCUMENT

### Patent Document

Korean Patent Registration No. 10-2287247, entitled “a composition of joint nutritional supplement for companion animals”
Korean Patent Registration No. 10-2463908 (Nov. 1, 2022), entitled “snacks for companion animals for improving intestinal health and a manufacturing method thereof”
Korean Patent Laid-Open Publication No. 10-2023-0083178 (Jun. 9, 2023), entitled “a composition for improving palatability and intestinal health of companion animals including sunflower lecithin”

### SUMMARY

The present invention was created to review and solve such various problems in the prior art as described above, and a main object of the present invention is to provide a composition for improving immunity of companion animals, which includes barley sprout extract powder, broccoli sprout extract powder, propolis, omega-3 fatty acid, zeolite powder, arginine powder, glutathione powder, chlorella powder, green leaf mussel extract powder, cranberry powder, shiitake powder, Sparassis crispa powder, Rich-Soybean paste power, brown rice (unpolished rice) enzyme powder, Kamut enzyme powder, plantago seed (psyllium) husk powder, Aspergillus oryzae powder, Monascus pilosus powder, Coenzyme Q-10 powder, zinc powder, N-acetylglucosamine, disodium EDTA and croscarmellose sodium as active ingredients, and enhances proliferation ability of peripheral blood mononuclear cells (PBMCs) through edible feeding to increase immunity, thereby contributing to health improvement.

As a means for achieving the above object, the present invention provides a composition for improving immunity of companion animals, which includes: 20 to 30 parts by weight (“wt. parts”) of barley sprout extract powder, 3 to 5 wt. parts of propolis, 5 to 15 wt. parts of omega-3 fatty acid, 1 to 2 wt. parts of zeolite powder, 5 to 10 wt. parts of glutathione powder, 3 to 5 wt. parts of chlorella powder, 5 to 10 wt. parts of green leaf mussel extract powder, 3 to 5 wt. parts of shiitake powder, 3 to 5 wt. parts of Sparassis crispa powder, 20 to 30 wt. parts of brown rice (unpolished rice) enzyme powder, 5 to 10 wt. parts of Kamut enzyme powder, 20 to 30 wt. parts of plantago seed (psyllium) husk powder, 3 to 5 wt. parts of Aspergillus oryzae powder, 3 to 5 wt. parts of Monascus pilosus powder, 3 to 5 wt. parts of Coenzyme Q-10 powder, 3 to 5 wt. parts of zinc powder, 3 to 5 wt. parts of N-acetylglucosamine, 3 to 5 wt. parts of disodium EDTA, and 3 to 5 wt. parts of croscarmellose sodium, based on 100 wt. parts of purified water.

In this case, the barley sprout extract powder may use powder prepared by the processes of: mixing 60 to 70 wt. parts of barley sprout and 30 to 40 wt. parts of biotin powder based on 100 wt. parts of the purified water and soaking the mixture for 3 to 4 hours, followed by steaming the same for 3 hours; after cooling to room temperature, placing the product in a fermentation chamber, introducing aerobic microorganisms thereto, fermenting the same for 3 days; and then drying and pulverizing the same.

In addition, the sprout broccoli extract powder may use powder prepared by the processes of: mixing 50 to 60 wt. parts of broccoli sprout, 15 to 20 wt. parts of green plum powder and 25 to 30 wt. parts of linseed oil based on 100 wt. parts of purified water; steaming the mixture for 2 hours, cooling the same to room temperature, and immersing the cooled product in diluted water containing vanadium ions diluted in water at a weight ratio of 1:1000 for 40 to 60 minutes; taking the soaked product out of the diluted water, placing the same in a fermentation chamber, and introducing aerobic microorganisms thereto, followed by fermenting for 2 days; and then drying and pulverizing the same.

Further, the brown rice enzyme powder may use powder prepared by the processes of: mixing 60 wt. parts of brown rice, 2 wt. parts of turmeric, 28 wt. parts of sodium carboxymethyl cellulose and 10 wt. parts of glycerin esters of fatty acids based on 100 wt. parts of purified water; steaming the mixture for 2 hours, and cooling the same to room temperature; placing the cooled product in a fermentation chamber, followed by introducing aerobic microorganisms thereto and fermenting the same for 2 days; and then drying and pulverizing the same.

According to the present invention, the composition of the present invention may include barley sprout extract powder, broccoli sprout extract powder, propolis, omega-3 fatty acid, zeolite powder, arginine powder, glutathione powder, chlorella powder, green leaf mussel extract powder, cranberry powder, shiitake powder, Sparassis crispa powder, Rich-Soybean paste power, brown rice (unpolished rice) enzyme powder, Kamut enzyme powder, plantago seed (psyllium) husk powder, Aspergillus oryzae powder, Monascus pilosus powder, Coenzyme Q-10 powder, zinc powder, N-acetylglucosamine, disodium EDTA and croscarmellose sodium as active ingredients, and enhance proliferation ability of peripheral blood mononuclear cells (PBMCs) through edible feeding to increase immunity, thereby attaining improved effects so as to contribute to health improvement.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a table showing results of evaluating changes in body weight according to the feeding of tablets of the present invention;

FIG. 2 is a table showing results of evaluating changes in feed intake according to the feeding of tablets of the present invention;

FIG. 3 is a table showing results of evaluating trend for amount of drinking water according to the feeding of tablets of the present invention;

FIG. 4 is a table showing results of evaluating skin and/or respiratory organ problems according to the feeding of tablets of the present invention;

FIG. 5 is a table showing results of hematological analysis according to the feeding of tablets of the present invention;

FIG. 6 is a table showing results of evaluating cell safety according to the feeding of tablets of the present invention;

FIG. 7 is photographs showing dogs of control and experimental groups according to the present invention;

FIG. 8 is a table illustrating results of lymphoid proliferation ability of the control and experimental groups according to the present invention; and

FIG. 9 is a table illustrating expression levels of cytokine according to the present invention.

### DETAILED DESCRIPTION

Hereinafter, preferred embodiments of the present invention will be described in detail with reference to the accompanying drawings.

Prior to the description of the present invention, the following specific structural or functional descriptions are only exemplified for the purpose of describing embodiments according to the concept of the present invention, and such embodiments according to the concept of the present invention may be implemented in various forms, and should not be construed as being limited to the embodiments described herein.

Then composition for improving immunity of companion animals according to the present invention includes barley sprout extract powder, broccoli sprout extract powder, propolis, omega-3 fatty acid, zeolite powder, arginine powder, glutathione powder, chlorella powder, green leaf mussel extract powder, cranberry powder, shiitake powder, Sparassis crispa powder, Rich-Soybean paste power, brown rice (unpolished rice) enzyme powder, Kamut enzyme powder, plantago seed (psyllium) husk powder, Aspergillus oryzae powder, Monascus pilosus powder, Coenzyme Q-10 powder, zinc powder, N-acetylglucosamine, disodium EDTA and croscarmellose sodium as active ingredients.

Therefore, these compositions may be dissolved in purified water and produced in the form of tablets, which in turn may be fed to a companion animal, in particular, a puppy.

More particularly, the composition of the present invention includes 20 to 30 wt. parts of barley sprout extract powder, 20 to 30 wt. parts of broccoli sprout extract powder, 3 to 5 wt. parts of propolis, 5 to 15 wt. parts of omega-3 fatty acid, 1 to 2 wt. parts of zeolite powder, 5 to 8 wt. parts of arginine powder, 5 to 10 wt. parts of glutathione powder, 3 to 5 wt. parts of chlorella powder, 5 to 10 wt. parts of green leaf mussel extract powder, 3 to 5 wt. parts of cranberry powder, 3 to 5 wt. parts of shiitake powder, 3 to 5 wt. parts of Sparassis crispa powder, 3 to 5 wt. parts of Rich-Soybean paste power, 20 to 30 wt. parts of brown rice (unpolished rice) enzyme powder, 5 to 10 wt. parts of Kamut enzyme powder, 20 to 30 wt. parts of plantago seed (psyllium) husk powder, 3 to 5 wt. parts of Aspergillus oryzae powder, 3 to 5 wt. parts of Monascus pilosus powder, 3 to 5 wt. parts of Coenzyme Q-10 powder, and 3 to 5 wt. parts of zinc powder, 3 to 5 wt. parts of N-acetylglucosamine, 3 to 5 wt. parts of disodium EDTA, and 3 to 5 wt. parts of croscarmellose sodium, based on 100 wt. parts of purified water.

In this case, for another example, the composition may be prepared while excluding broccoli sprout extract powder, arginine powder, cranberry powder and Rich-Soybean paste power.

In addition, for another example, the composition may be prepared while excluding glutathione powder, Kamut enzyme powder, Aspergillus oryzae powder and zinc powder.

At this time, the barley sprout extract powder contains large amounts of beta-carotene, flavonol glycoside rutin, selenium, crystalline polypeptide glutathione and quercetin, which are antioxidants, thereby contributing to improvement of intestinal health.

Further, the broccoli sprout extract powder contains a large amount of sulforaphane component, thereby contributing to adjustment of blood glucose and enhancement of intestinal health.

In addition, the propolis consists of diverse compounds including more than 500 species. Among them, flavonoid is the most effective and important active ingredient, which is known to contribute to anti-inflammation, anti-oxidation, immune enhancement and the like.

Further, the omega-3 fatty acid is a type of unsaturated fatty acids and an essential fatty acid necessarily required for the human body, however, cannot be synthesized in vivo thus should be replenished in the form of food or supplements.

It has been reported that such omega-3 fatty acid serves to the reduction of blood triglyceride, improved inflammation and mitigation of ulcerative colitis, and effectively proliferates intestinal beneficial bacteria.

Further, the zeolite powder serves to purification or antioxidation by far-infrared radiation.

Additionally, the arginine powder is one of twenty (20) types of amino acids to form a protein, and mostly contained in large content in herrings and salmons.

Such arginine may enhance blood flow, reduce inflammation, and in particular, contribute to intestinal health of companion animals.

In addition, the glutathione powder may serve to cell proliferation, immune reaction and inhibition of oxidation stress due to anti-oxidation. Most of all, it is known to exhibit effects in enhancement of immunity through activation of intestinal beneficial bacteria.

Further, the chlorella powder is a powdered product of a type of planktons, is rich in vitamins and minerals, and in particular, contains carotenoid as an antioxidant such that it is helpful for not only enhancing immunity but also improving intestinal health of companion animals.

Furthermore, the green leaf mussel extract powder contains large amounts of omega-3 fatty acid, fatty acid chondroitin and sulfate, therefore, may serve to implement anti-oxidation effects, inflammation inhibition, activation of intestinal beneficial bacteria in companion animals, thereby contributing to enhancement of immunity.

In addition, due to a large amount of proanthocyanidine contained in the cranberry powder, adhesion of Escherichia coli causing urinary tract infection to epithelial cells is inhibited, acidity of urine is increased to remove bacteria, and anti-inflammatory effects are exhibited. These functions are expressed through activation of immune cells.

Further, the shiitake powder contains large amounts of beta-glucan, lentinan, vitamin D components, etc. United States Food and Drug Administration (FDA) had announced that these components have excellent efficacy in immunity reinforcement and anticancer action. In particular, the lentinan component has been reported as one among representative anticancer components to inhibit development and propagation of cancer cells.

Moreover, the Sparassis crispa powder is a food containing beta-glucan the most, wherein the beta-glucan promotes secretion of a substance called cytokine to increase immunity, and serves to make the body healthy.

Further, the Rich-Soybean paste power is rich in fibers and contains bacillus bacteria to have excellent intestinal regulation. In particular, it contains genistein, saponin and phytic acid to provide strong anti-oxidative effects and immunity reinforcing effects, while isoflavone has been reported to remove active oxygen and thus is effective in preventing cancer.

Furthermore, the brown rice (unpolished rice) enzyme powder is rich in dietary fibers to facilitate improvement of intestinal health, and also serves to promote digestion and activation of intestinal microorganisms.

Particularly, the phytic acid contained therein suppresses active oxygen and serves to increase anti-oxidation.

Further, the Kamut enzyme powder has strong anti-oxidation due to selenium and thus excellent anticancer effects, and is effective in improving and treating inflammation, such that intestinal microorganism environments become favorable, thereby contributing to increase in beneficial bacteria for improving immunity.

Furthermore, the plantago seed (psyllium) husk powder is obtained by pulverizing the husk of plantain seeds, which can promote absorption of moisture in the bowel to improve constipation, reduce cholesterol, inhibit active oxygen, and serve to activation of intestinal microorganisms.

Moreover, the Aspergillus oryzae powder is a type of yeasts used for fermenting alcoholic drink and one of immunity reinforcing substances, which can inhibit intestinal harmful bacteria and facilitate proliferation of beneficial bacteria, thereby retaining intestinal health.

Further, the Monascus pilosus powder has been reported to improve a blood cholesterol level, so as to decrease blood pressure and enhance insulin resistance and diabetes, while increasing immunity thus to efficiently inhibit inflammation.

Moreover, the Coenzyme Q-10 has superior effects of removing active oxygen, therefore, is now broadly used all around the world in order to prevent and treat cardiovascular diseases, reinforce immune function and for anti-aging use.

Further, the zinc powder is an important component to recover functions of immunocytes through activation of macrophages and NK cells.

Further, the N-acetylglucosamine is obtained by hydrolyzing chitin, an ingredient forming the shell of crustaceans such as shrimp and crabs, and suppresses aging by reducing e activity for collagen decomposition and elastin decomposition, and thereby, above all, contributing to enhancing the defense function against bacterial invasion.

In addition, the disodium EDTA is a water-soluble ingredient bound to two sodium ions in the existing EDTA ingredient and performs functions to prevent rancidity due to oxidation.

Furthermore, the croscarmellose sodium is obtained by hydrolyzing peptide with acids, enzymes, or other methods, and contributes to improvement of antibacterial efficacy and immunity, and reinforcement of skin barrier.

In addition, in the present invention, 10 wt. parts of inulin powder and 15 wt. parts of biotin powder may be further added based on 100 wt. parts of the purified water.

In this case, the inulin powder is obtained by washing and drying hypogeal stems of Chrysantemum, bulbs of Dahlia, Burdock roots, etc., and then pulverizing the dried plants, which performs functions of Prebiotics, for example, is fermented in the colon and promotes the growth of beneficial microorganisms to prevent or delay the growth of spoilage microorganisms and maintain the intestinal health, and thereby reinforcing the immunity.

Further, the biotin powder is obtained by pulverizing sulfur-containing water soluble vitamins, which is degraded during fermentation and participates in lipid synthesis, protein and/or hydrocarbonate metabolism, thereby contributing to enhancement of intestinal health of companion animals.

Moreover, in the present invention, 10 wt. parts of apple juice, 10 wt. parts of sodium carboxymethyl cellulose and 10 wt. parts of burdock powder may be further added based on 100 wt. parts of the purified water.

In this case, the apple juice is rich in pectin and cellulose to prevent gastroenteric trouble, keeps the intestine slightly acid to inhibit harmful bacteria while preparing an environment good for breeding beneficial bacteria, thereby contributing to enhancing immunity.

Further, the sodium carboxymethyl cellulose is water-soluble cellulose and thus serves to improve liver diseases through fibers.

In addition, the burdock powder is rich in dietary fibers and fructo-oligosaccharide and thus acts similar to lactobacillus in the intestine to improve functions of the colon and kidney, and facilitates diuretic action to excrete waste from the body, thereby contributing to health improvement.

Furthermore, in the present invention, 10 wt. parts of soybean powder and 10 wt. parts of trehalose may be further added based on 100 wt. parts of the purified water.

In this case, the soybean powder is a representative phytochemical, wherein isoflavone is contained in a large amount to remove active oxygen and efficiently prevent cancer, and is known to greatly contribute to reinforcement of intestinal immunity.

In addition, the trehalose is a disaccharide composed of two glucose molecules, which is used in a liquid state dissolved in water, and serves to enhance sweetness and stabilize pH.

Meanwhile, the barley sprout extract powder uses powder prepared by the processes of: mixing 60 to 70 wt. parts of barley sprout and 30 to 40 wt. parts of biotin powder based on 100 wt. parts of the purified water and soaking the mixture for 3 to 4 hours, followed by steaming the same for 3 hours; after cooling to room temperature, placing the product in a fermentation chamber, introducing aerobic microorganisms thereto, fermenting the same for 3 days; and then drying and pulverizing the same.

Further, the broccoli sprout extract powder uses powder prepared by the processes of: mixing 50 to 60 wt. parts of broccoli sprout, 15 to 20 wt. parts of green plum powder and 25 to 30 wt. parts of linseed oil based on 100 wt. parts of purified water; steaming the mixture for 2 hours, cooling the same to room temperature, and immersing the cooled product in diluted water containing vanadium ions diluted in water at a weight ratio of 1:1000 for 40 to 60 minutes; taking the soaked product out of the diluted water, placing the same in a fermentation chamber, and introducing aerobic microorganisms thereto, followed by fermenting for 2 days; and then drying and pulverizing the same.

At this time, the purpose of using the diluted water is to improve vascular health of the companion animal so as to induce inhibition of dermatitis, sedative action and astriction by absorbing vanadium as a natural mineral and nutrient, which is harmless to the companion animal, in broccoli and then fermenting the same.

In particular, the green plum powder and linseed oil overcome indigestion and serve to activity of intestinal beneficial bacteria, thereby contributing to improvement of constipation, promotion of evacuation, and enhancement of immunity.

Further, the brown rice (unpolished rice) enzyme powder uses powder prepared by the processes of: mixing 60 wt. parts of brown rice, 2 wt. parts of turmeric, 28 wt. parts of sodium carboxymethyl cellulose and 10 wt. parts of glycerin esters of fatty acids based on 100 wt. parts of purified water; steaming the mixture for 2 hours, and cooling the same to room temperature; placing the cooled product in a fermentation chamber, followed by introducing aerobic microorganisms thereto and fermenting the same for 2 days; and then drying and pulverizing the same.

At this time, the sodium carboxymethyl cellulose is water-soluble cellulose and serves to improve intestinal diseases through fibers.

Further, the glycerin esters of fatty acids are generally added to ensure emulsification and easy intake by the companion animals.

Further, the plantago seed (psyllium) husk powder uses powder prepared by the processes of: mixing 60 wt. parts of psyllium husk and 15 wt. parts of Winding snoutbean peptide based on 100 wt. parts of purified water; steaming the mixture for 2 hours, and cooling the same to room temperature; placing the cooled product in a fermentation chamber, followed by introducing aerobic microorganisms, and fermenting the same for 2 days; and then drying and pulverizing the same.

In this case, the Winding snoutbean peptide contains glycitein so as to exhibit various efficacies such as detoxication, diuretic action, waste removal, inhibition of cholesterol, etc. through hormone control, therefore, contributes to intestinal health and is helpful for improvement of blood circulation of companion animals.

In this regard, powdery agar may be added in an amount of 15 wt. parts based on 100 wt. parts of the purified water used for preparing the plantago seed (psyllium) husk powder, wherein the powdery agar is seldom corrupted while having high affinity to water. Further, the powdery agar contains large amounts of protein, calcium, fibrin (cellulose), phosphorous, iron and vitamin C, therefore, may improve constipation, induce satiety, and is effective in improving intestinal health of companion animals.

Further, Hedyotis diffusa Willd extract may be added in an amount of 15 wt. parts based on 100 wt. parts of the purified water used for preparing the plantago seed (psyllium) husk powder, wherein the Hedyotis diffusa Willd extract detoxifies intestinal toxin of the companion animal, treats inflammation, and serves to enhancement of immune function.

Hereinafter, examples will be described.

### Example 1

First, the composition for improving immunity of companion animals according to the present invention was prepared as follows, and then soaked in purified water to form a formulation in a tablet form so that it could be fed to companion animals, especially puppies.

That is, based on 100 wt. parts of purified water, 25 wt. parts of barley sprout extract powder, 25 wt. parts of broccoli sprout extract powder, 4 wt. parts of propolis, 10 wt. parts of omega-3 fatty acid, 1 wt. parts of zeolite powder, 6 wt. parts of arginine powder, 7 wt. parts of glutathione powder, 4 wt. parts of chlorella powder, 7 wt. parts of green leaf mussel extract powder, 4 wt. parts of cranberry powder, 4 wt. parts of shiitake powder, 4 wt. parts of Sparassis crispa powder, 4 wt. parts of Rich-Soybean paste power, 25 wt. parts of brown rice (unpolished rice) enzyme powder, 7 wt. parts of Kamut enzyme powder, 25 wt. parts of plantago seed (psyllium) husk powder, 4 wt. parts of Aspergillus oryzae powder, 4 wt. parts of Monascus pilosus powder, 4 wt. parts of Coenzyme Q-10 powder, and 4 wt. parts of zinc powder, 4 wt. parts of N-acetylglucosamine, 4 wt. parts of disodium EDTA and 4 wt. parts of croscarmellose sodium were mixed together to form a formulation in a tablet form.

### Example 2

The same procedure as in Example 1 was conducted, except that the barley sprout extract powder was prepared by mixing 70 wt. parts of barley sprout and 30 wt. parts of biotin powder with 100 wt. parts of purified water, soaked for 3 hours and then steamed for 3 hours. Then, after cooling to room temperature, the treated mixture was placed in a fermentation chamber and aerobic microorganisms were introduced thereto, followed by fermentation for 3 days, and then drying and pulverizing the same to produce desired powder, which in turn was used.

### Example 3

The same procedure as in Example 2 was conducted, except that the broccoli sprout extract powder was prepared by mixing 60 wt. parts of broccoli sprout, 15 wt. parts of green plum powder and 25 wt. parts of linseed oil were mixed with 100 wt. parts of purified water, steamed for 2 hours, and then cooled to room temperature. Then, after immersing the mixture in diluted water containing vanadium ions diluted in water at a weight ratio of 1:1000 for 40 minutes, the mixture was taken and placed in a fermentation chamber, and aerobic microorganisms were introduced thereto, followed by fermentation for 2 days, and then drying and pulverizing the same to produce desired powder, which in turn was used.

### Example 4

The same procedure as in Example 3 was conducted, except that the brown rice (unpolished rice) enzyme powder was prepared by mixing 60 wt. parts of brown rice, 2 wt. parts of turmeric, 28 wt. parts of sodium carboxymethyl cellulose and 10 wt. parts of glycerin esters of fatty acids were mixed with 100 wt. parts of purified water, steamed for 2 hours, and then cooled to room temperature. Then, after placing the cooled product in a fermentation chamber, aerobic microorganisms were introduced thereto, followed by fermentation for 2 days, and then drying and pulverizing the same to produce desired powder, which in turn was used.

### Example 5

The same procedure as in Example 4 was conducted, except that the plantago seed (psyllium) husk powder was prepared by mixing 60 wt. parts of psyllium, 15 wt. parts of Winding snoutbean peptide, 15 wt. parts of powdery agar, and 10 wt parts of Hedyotis diffusa Willd were mixed with 100 wt. parts of purified water, steamed for 2 hours, and cooled to room temperature. Then, after placing the cooled product in a fermentation chamber, aerobic microorganisms were introduced thereto, followed by fermentation for 2 days, and then drying and pulverizing the same to produce desired powder, which in turn was used.

Then, the oral administration safety of the tablets of [Examples 1-5] prepared as described above was assessed.

The oral administration safety assessment was performed by evaluating the average change in body weight, feed intake, water intake, skin and whether or not respiratory problems occur for the five examples while feeding the composition of the present invention (Examples 1-5). The composition was processed in the form of tablets, and experiments were conducted for 6 weeks with feeding once a day. The experimental subjects were divided into a control group, a clinical dose group, and a clinical dose 3-fold group for 6 adult 5-year-old pet dog beagles, and tested for changes in the body weight while feeding the tablets of Examples 1-5, respectively. The same living environment conditions were set, and experiment results are shown in the drawings.

### [Evaluation of Changes in Body Weight]

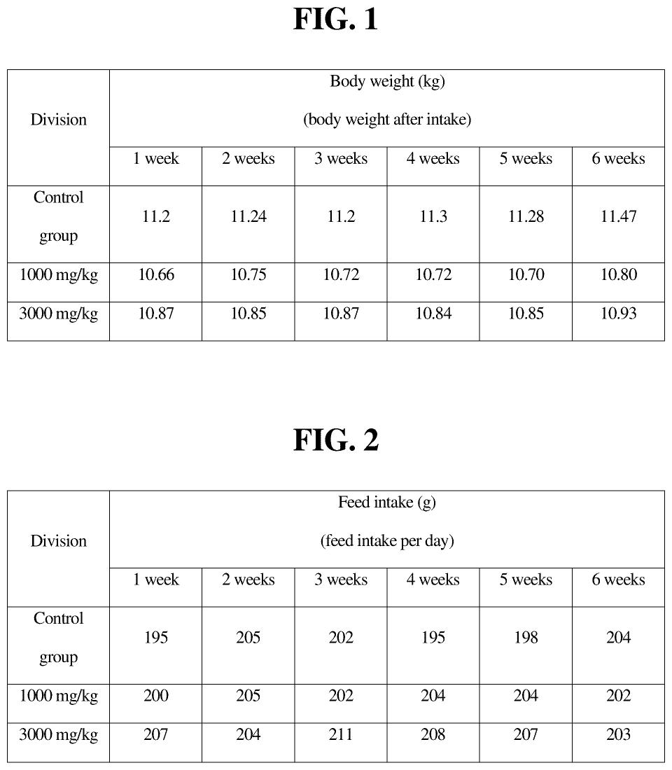

As shown in FIG. 1, in the case of the control group fed with the composition of the present invention, the initial body weight was observed as 11.2 kg while having 11.47 kg at the end of the experiment. On the other hand, for the clinical dose group (1000 mg/kg), it was confirmed to have 10.66 kg at the beginning and 10.80 kg at the end, and for the clinical dose 3-fold group (3000 mg/kg), it was confirmed to have 10.87 kg at the beginning and 10.93 kg at the end.

That is, rapid change in body weight or abnormal symptoms according to the feeding of the composition of the present invention were not found in all of the control group, clinical dose group, and clinical dose 3-fold group, therefore, the composition of the present invention was determined to be safe.

### [Evaluation of Feed Intake]

As shown in FIG. 2, in the case of feed intake, the control group was observed to have 195 g at the beginning and 204 g at the end of the experiment, and the clinical dose group was observed to have 200 g at the beginning and 202 g at the end of the experiment. Further, it was confirmed that the clinical 3-fold dose group has the initial intake of 207 g and 203 g at the end of the experiment. Therefore, it was determined that the feed intake of the clinical 3-fold dose group was not significantly reduced compared to the control group.

That is, rapid change in feed intake or abnormal symptoms according to the feeding of the composition of the present invention were not found in all of the control group, clinical dose group, and clinical 3-fold dose group.

### [Evaluation of Trend for Amount of Drinking Water]

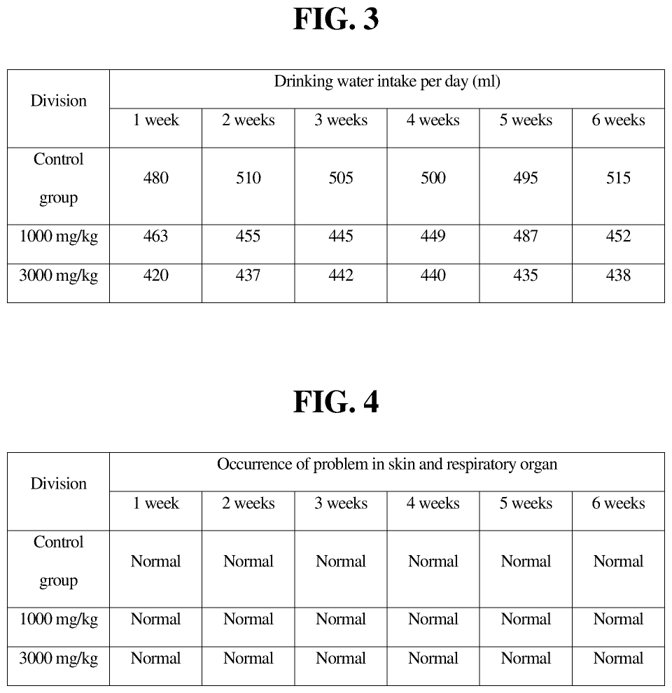

As shown in FIG. 3, in the case of the amount of drinking water, the control group was observed to have 480 ml at the beginning and 515 ml at the end of the experiment, the clinical dose group was observed to have 463 ml at the beginning and 452 ml at the end of the experiment, and the clinical 3-fold dose group was determined to have 420 ml at the beginning and 438 ml at the end of the experiment.

Therefore, it could be confirmed that the amount of drinking water also did not shown a significant change compared to the control group. In addition, skin, respiratory, behavior, etc. were evaluated for abnormal symptoms, but no significant abnormal symptoms were found.

### [Evaluation of Problems in Skin and Respiratory Organ]

As shown in FIG. 4, no rapid decrease or increase in body weight caused by oral administration of the composition of the present invention was observed, or abnormal changes in food intake, and abnormal changes in amount of drinking water were not found. Further, skin rash, dyspnoea, pathogenesis, and abnormal behavior due to stress were not observed.

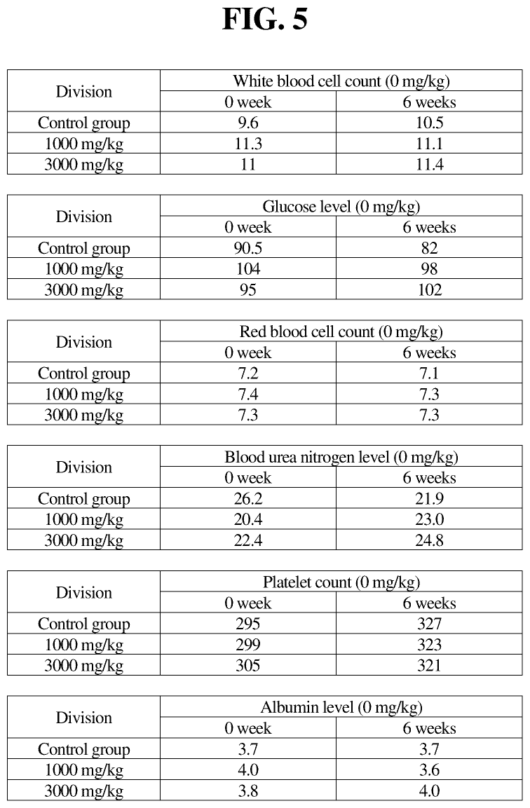

Further, the safety of oral administration was evaluated by analyzing hematology parameters such as white blood cell (WBC), red blood cell (RBC), and platelet (PLT) counts, and blood chemistry parameters such as glucose (GLC), blood urea nitrogen (BUN), and albumin (ALB) levels. Results thereof are shown in FIG. 5. Further, the change is expressed as an average value while feeding Examples 1-5 for each pet dog.

Meanwhile, as shown in FIG. 5, as a result of observing changes in white blood cell count, red blood cell count, platelet count, etc. through hematological analysis, all of the values were determined to be within the normal range and no significant abnormal results were observed. Although the glucose levels, blood urea nitrogen levels, and albumin levels were tested to assess liver, kidney and internal organ abnormalities through blood chemistry analysis, no enzyme changes were observed in the kidneys, hepatobiliary system, and internal organs. Therefore, it was determined that there is no problem with oral administration of the composition of the present invention.

### [Evaluation of Cell Safety]

In order to assess the safety, cell viability was measured, which was conducted by applying the method of Carmichael et al. Specifically, the incubated cells were dispensed by 2×10²/well in a 96-well plate, and after 24 hours, the composition of the present invention was diluted in PBS or 50% DMSO as a solvent at 200 and 600 μg/mL, followed by culturing for 24 hours. Thereafter, 200 μL of 1 mg/mL MTT solution was added thereto, followed by culturing for 3 hours, and then the supernatant was removed.

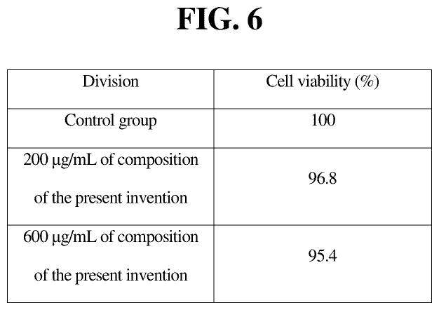

Thereafter, 200 μL DMSO was added thereto, followed by agitation in a dark room for 30 minutes. Then, absorbance was measured at 540 nM wavelength by a microplate reader (BioTek Winiiski, USA) in order to estimate the cell viability, i.e., cell survival rate. Results thereof are shown in FIG. 6.

The cell viability was converted relative to 100% of the control group without drug treatment (CON). When the cell viability is 80% or less, it was determined as a concentration with toxicity.

The composition of the present invention showed the cell viability of 95% or more. Based on these results, the composition of the present invention was determined to have no cytotoxicity, which in turn could be analyzed to ensure safety.

Meanwhile, in order to confirm whether to improve immunity or not by feeding the companion animal (e.g., puppy) with the composition according to the present invention, peripheral blood mononuclear cell (PBMC) lymphoid proliferation ability was evaluated.

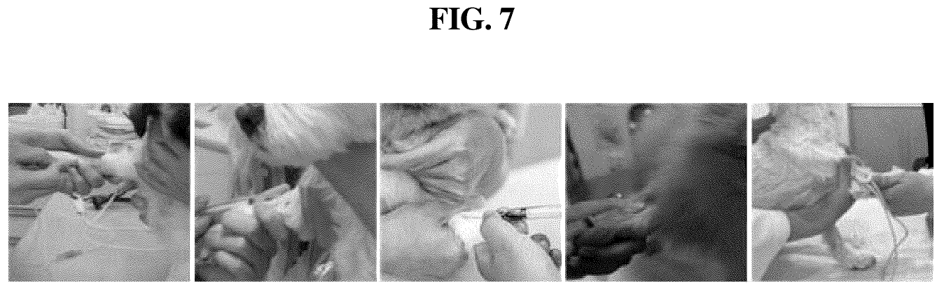

For subject companion animals including one control puppy and five experimental puppies at Abandoned Pet Protection Center, the experiment was conducted under an agreement and consent for hereafter treatment support and humanitarian aid, and experiment-related photographs are shown in FIG. 7.

As shown in FIG. 7, the corresponding control group and experimental groups were divided and accepted in separate and independent dog houses. The animals were fed with the same feed and plenty of water, and provided with an environment without external parameters for experiment.

Further, vaccination against canine distemper virus, parvovirus, etc. was completed for the experimental dogs, followed by the experiment. Among them, the dogs aged 5 or more having received antibody titer test were selected as test subjects. The subjects were fed at 16 hour once a day by 1000 mg/kg of body weight and, for easy oral administration, the feed was mixed with microcrystalline cellulose at a final mixing ratio of 1:1 and then fed in the form of tablets.

Furthermore, conditions of the experimental groups were checked in advance to confirm that the animals did not have any alternative disease, and then, the experiment was conducted.

(1) Evaluation of Peripheral Blood Mononuclear Cell (PBMC) Lymphoid Proliferation Ability (a Method for Confirming Whether to Reinforce Immunity or not)

The peripheral blood mononuclear cells (PBMCs) are blood cells having a rounded nucleus and composed of lymphoids (T cell, B cell and NK cell) as well as monocytes. Meanwhile, ficoll is a hydrophilic polysaccharide as a substance that can separate the blood into a plasma component in the supernatant, PBMC, polymorphonuclear cells in the lowermost portion (e.g., neutrophil and eosinophil), and red blood cells.

After oral administration of the composition of the present invention, blood was collected at weeks 0, 5 and 10 after fasting for 12 hours, respectively. Then, in order to separate the peripheral blood mononuclear cells, 5 ml of blood and the same amount of phosphate buffered saline (PBS) (a type of balanced salines, which is a solution including physiological saline added with phosphate so as to further stably maintain pH) were mixed together, and then this mixture was floated on a 50 ml tube containing a substance for forming a density slope, that is, 15 ml of ficoll in order to separate the cells, followed by centrifugation of the tube containing the sample (1500 rpm/20 min).

For washing the isolated cells, the cells were moved to a tube containing fresh PBS solution and centrifuged (1500 rpm/15 min). The isolated PBMCs were mixed with a growth medium RPMI 1640 (Roswell Park Memorial Institute-1640 medium, Thermo Fisher Scientific) used for cell incubation, and 20% of fetal bovine serum (FBS, PAN biotech company) added and used in the culture of mammal cells.

In addition, PBMCs isolated from the experimental group blood were seeded on 96-well plates, and incubated for 1 week in a CO₂ incubator (at 37° C. with 5% humidity) together with 200 ul of 20% fetal bovine serum (FBS), and 10 ng/ml of recombinant canine interleukin-2 (IL-2) protein (Yongin R&D).

Further, 100 μL of cell supernatant was discarded at an interval of 2 to 3 days, and then 200 μL of fresh cell culture and growth medium RPMI-1640 and 100 μL of a mixture containing recombinant canine IL-2 protein and 20% FBS were added thereto.

In addition, absorbance was measured at 450 nm to confirm the proliferation ability using a cell viability assay kit (Medifab) according to the manual provided by the manufacturer.

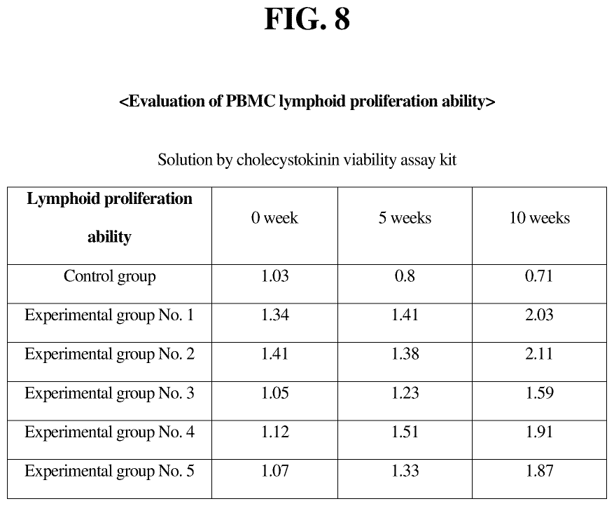

In addition, evaluation results are shown in FIG. 8.

As shown in FIG. 8, the control group without administration of the composition of the present invention had the lymphoid proliferation ability of 1.03 at week 0 after administration, while experimental group No. 1 with administration of the composition of the present invention by 1000 mg/l kg of body weight showed 1.34. Similarly, experimental group No. 2 had 1.41, experimental group No. 3 had 1.05, experimental group No. 4 had 1.12, and experimental group No. 5 had 1.07. Further, the lymphoid proliferation ability of the control group was 0.8 at week 5 after administration, while experimental group No. 1 showed 1.41, experimental group No. 2 showed 1.38, experimental group No. 3 showed 1.23, experimental group No. 4 showed 1.51, and experimental group No. 5 showed 1.33. Further, at week 10 after administration, the lymphoid proliferation ability of the control group was 0.71, while experimental group No. 1 showed 2.03, experimental group No. 2 showed 2.11, experimental group No. 3 showed 1.59, experimental group No. 4 showed 1.91, and experimental group No. 5 showed 1.87, whereby the experimental groups demonstrated a tendency of increase in proliferation ability as compared to the experimental groups at week 0 after administration. In addition, it could be confirmed that, as compared to the control group, feeding the composition of the present invention may further increase proliferation ability of lymphoids (consisting of T cell, B cell and NK cell) with immune action.

### (2) Evaluation of TNF Alpha, IL-1 Beta Expression Levels by Lipidpolysaccharide (LPS) Stimulation

PBMCs isolated from the control and experimental group bloods entered a mixture containing 200 μL RPMI-1640 cell culture medium and 200 μL of 20% FBS in a 96-well plate. Further, the prepared mixture was subjected to stimulation using recombinant canine IL-2 protein and lipopolysaccharides (LPS, 50 ng/mL) (amphipathic molecules including fatty acid and polysaccharides combined therein, which is a representative cause of inducing inflammation to express toxicity) to culture the same. From the cultured PBMC supernatant, canine tumor necrosis factor-alpha (TNF alpha) and interleukin-1 beta (IL-1 beta) were measured using Elisa kit, respectively.

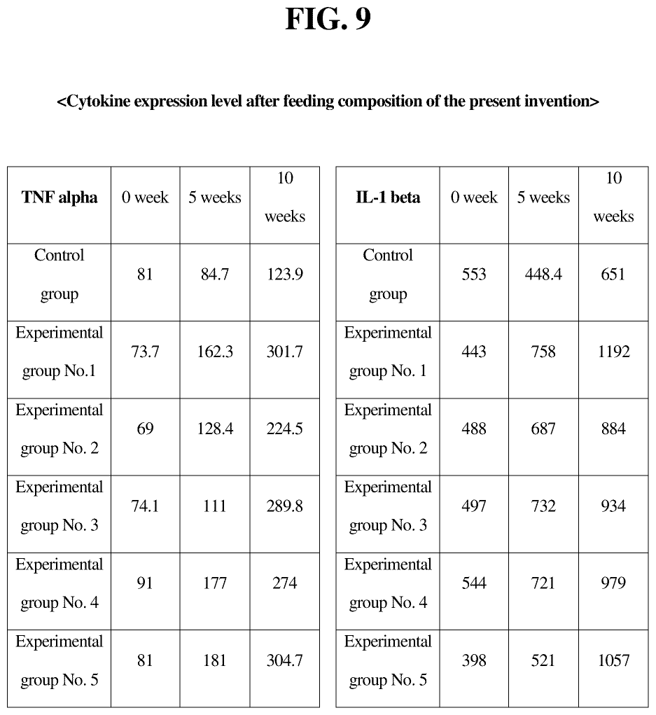

At this time, an expression level of cytokine was the same as those shown in FIG. 9.

As shown in FIG. 9, TNF alpha of the control group was 81 at week 0 after administration, while experimental group No. 1 showed 73.7, experimental group No. 2 showed 69, experimental group No. 3 showed 74.1, experimental group No. 4 showed 91, experimental group No. 5 showed 81. Further, at week 5 after administration, the control group had 84.7 while experimental group No. 1 showed 162.3, experimental group No. 2 showed 128.4, experimental group No. 3 showed 111, experimental group No. 4 showed 177, and experimental group No. 5 showed 181. Further, at week 10 after administration, the control group had 123.9, while experimental group No. 1 showed 301.7, experimental group No. 2 showed 224.5, experimental group No. 3 showed 289.8, experimental group No. 4 showed 274, and experimental group No. 5 showed 304.7, whereby significant differences in TNF alpha production could be confirmed.

A major role of TNF alpha is to adjust immune cells, specifically may act as a heating source in vivo or induce apoptosis, etc. Further, TNF alpha is a tumor necrosis factor observed in an acute phase reaction during inflammatory responses. In the present example, it showed significant differences as compared to the control group, and this may be determined because lymphoids having immune response according to the feeding of the composition of the present invention are increased, which in turn causes an increase in TNF alpha secretion.

Further, in the case of the amount of IL-1 beta, it was seen that the control group at week 0 after administration had 533, experimental group No. 1 had 443, experimental group No. 2 had 488, experimental group No. 3 had 497, experimental group No. 4 had 544, and experimental group No. 5 had 398. In addition, at week 5 after administration, the control group showed 448.4, experimental group No. 1 showed 758, experimental group No. 2 showed 687, experimental group No. 3 showed 732, experimental group No. 4 showed 721, and experimental group No. 5 showed 521. Further, at week 10 after administration, the control group showed 651, experimental group No. 1 showed 1192, experimental group No. 2 showed 884, experimental group No. 3 showed 934, experimental group No. 4 showed 979, and experimental group No. 5 showed 1057, whereby significant differences in generation of IL-1 beta could be confirmed. IL-beta is a cytokine which is a major regulatory factor involved in infection and damage. IL-beta is generally and mostly produced in monocytes and macrophages. Accordingly, the experimental groups showed significant differences in generation of IL-1 beta as compared to the control group, and this may be determined because of effects of monocytes proliferated by the composition of the present invention.

(3) Evaluation of B Cell and T Cell Distribution after Feeding the Composition of the Present Invention

After feeding the composition of the present invention, peripheral blood mononuclear cells were isolated from bloods of beagles in an amount of 10⁵ cell/ml or more at weeks 0, 5 and 10, respectively, washed twice with phosphate buffered saline (PBS), followed by centrifugation (150 rpm/5 min) to suspend the same on 100 μl of PBS in different states.

50 μl of the solution was maintained in unstained state, while B cell marker, that is, cd21 antibody (a cell adhering molecule found on the surface of B cell) entered the remaining 50 μl of the solution and allowed to react on ice to be stained.

After washing the product twice with phosphate buffered saline (PBS) again, 2% paraformaldehyde (PFA) solution was added thereto to fix the same for 10 minutes. Next, after washing twice with PBS, the product was suspended by adding PBS and stored in a light-shielding state. Thereafter, the product was assessed using a FACSCalibur flow cytometer.

As a result of the assessment, the antibody assessed by flow cytometry (a method for analyzing physico-chemical features simultaneously by passing flowing cells through an electrical sensing device) showed that a ratio of B cells to T cells in each individual and group was similarly maintained without significant differences. Therefore, it could be confirmed that normal immunity is retained.

Consequently, peripheral blood mononuclear cells (PBMCs) are immune cells consisting of B cells, T cells, natural killer (NK) cells, and monocytes, so as to induce selective reactions in the immune system. A cell division promoting substance, motpgen may include, for example, concana-valin A (Con a), polyhydroxyalkanoates (PHAs) and lipidpolysaccharides (LPSs), etc., which is a substance bound to a receptor group on a surface film of the cellular membrane of lymphoids to cause DNA synthesis and cell division of the corresponding cells. In Example 5-(1), according to the feeding of the <composition of the present invention>, significant differences in lymphoid proliferation ability due to mitogen stimulation were observed as compared to the control group, thus it could be seen that the composition of the present invention has influence on the proliferation ability of PBMCs serving to immune function.

1. A composition for improving immunity of companion animals, the composition comprising: 20 to 30 parts by weight (“wt. parts”) of barley sprout extract powder, 3 to 5 wt. parts of propolis, 5 to 15 wt. parts of omega-3 fatty acid, 1 to 2 wt. parts of zeolite powder, 5 to 10 wt. parts of glutathione powder, 3 to 5 wt. parts of chlorella powder, 5 to 10 wt. parts of green leaf mussel extract powder, 3 to 5 wt. parts of shiitake powder, 3 to 5 wt. parts of Sparassis crispa powder, 20 to 30 wt. parts of brown rice (unpolished rice) enzyme powder, 5 to 10 wt. parts of Kamut enzyme powder, 20 to 30 wt. parts of plantago seed (psyllium) husk powder, 3 to 5 wt. parts of Aspergillus oryzae powder, 3 to 5 wt. parts of Monascus pilosus powder, 3 to 5 wt. parts of Coenzyme Q-10 powder, 3 to 5 wt. parts of zinc powder, 3 to 5 wt. parts of N-acetylglucosamine, 3 to 5 wt. parts of disodium EDTA, and 3 to 5 wt. parts of croscarmellose sodium, based on 100 wt. parts of purified water.

2. The composition according to claim 1, wherein the barley sprout extract powder uses powder prepared by the processes of: mixing 60 to 70 wt. parts of barley sprout and 30 to 40 wt. parts of biotin powder based on 100 wt. parts of the purified water and soaking the mixture for 3 to 4 hours, followed by steaming the same for 3 hours; after cooling to room temperature, placing the product in a fermentation chamber, introducing aerobic microorganisms thereto, fermenting the same for 3 days; and then drying and pulverizing the same.

3. The composition according to claim 1, wherein the sprout broccoli extract powder uses powder prepared by the processes of: mixing 50 to 60 wt. parts of broccoli sprout, 15 to 20 wt. parts of green plum powder and 25 to 30 wt. parts of linseed oil based on 100 wt. parts of purified water; steaming the mixture for 2 hours, cooling the same to room temperature, and immersing the cooled product in diluted water containing vanadium ions diluted in water at a weight ratio of 1:1000 for 40 to 60 minutes; taking the soaked product out of the diluted water, placing the same in a fermentation chamber, and introducing aerobic microorganisms thereto, followed by fermenting for 2 days; and then drying and pulverizing the same.

4. The composition according to claim 1, wherein the brown rice enzyme powder uses powder prepared by the processes of: mixing 60 wt. parts of brown rice, 2 wt. parts of turmeric, 28 wt. parts of sodium carboxymethyl cellulose and 10 wt. parts of glycerin esters of fatty acids based on 100 wt. parts of purified water; steaming the mixture for 2 hours, and cooling the same to room temperature; placing the cooled product in a fermentation chamber, followed by introducing aerobic microorganisms thereto and fermenting the same for 2 days; and then drying and pulverizing the same.
